(19) United States
(12) Patent Application Publication
(10) Pub. No.: US 2004/0224878 A1
(43) Pub. Date: Nov. 11, 2004

(54) Fertilization of aged oocytes

(76) Inventors: Suna Cukurcam, Berlin (DE); Christa Hegele-Hartung, Mieheim/Ruhr (DE); Thomas Blume, Schildow (DE); Peter Esperling, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/276,399

(22) PCT Filed: May 9, 2001
(86) PCT No.: PCT/EP01/05285

(30) Foreign Application Priority Data
May 18, 2000 (EP) 00250154.2

(51) Int. Cl. A61K 38/18; A01N 1/02

(52) U.S. Cl. 514/12; 435/2

(57) ABSTRACT

The present invention relates to the use of FF-MAS and/or MAS analogues for the fertilization of aged ootcyts.

The invention relates to the use of meiosis-activating substances for the fertilization of aged oocytes.

The reproductive potential of a population is defined by its fecundity. Fecundity describes the monthly conceptions that naturally occur among sexually active couples. Little change in fecundity occurs until women reach their mid thirties when rates begin to fall. Rates are reduced by 50% in women over age 37, and as much as 95% by the age of 45% (1). Compared to the population at large, women over the age of 40 represent less than 1% of all mothers giving birth, and only 0.01% of deliveries occur in women 47 years and older (2). However, increasing numbers of older women in the Western world are attempting pregnancy (3). Underlying reasons for this increasing trend include later and second marriages, the pursuit of educational or vocational goals as well as delayed reproduction resulting from effective contraception.

A fixed store of oocytes is formed in the ovary before birth and is progressively depleted thereafter. The numbers of non-ovulated oocytes decline at a steady rate until approx. age 37, after which the process accelerates 2- to 3-fold. Therefore, a state of “reproductive menopause” exists approx. 10 years before the cessation of menses indicates the onset of the “endocrine menopause” (4).

Decline of the oocyte number is due to atresia and increases more than twofold after the age of 37, ultimately exhausting the store of gametes (5). The reduction in oocyte number with age is further accelerated by a raise of the remaining oocytes into the growing stages, which regularly occurs before ovulation. This increased cohort of growing oocytes will degenerate more rapidly after a dominant oocyte is selected (6).

In the remaining oocytes of older women, which may have the potential to be fertilised in vivo or in vitro, an altered meiotic cell cycle with spindle and cytoskeletal aberrations as well as an impaired function of mitochondria is often observed (7). These alterations in the meiotic cell cycle may lead to precocious or delayed divisions of chromosomes. The loss of the capacity to faithfully separate chromosomes with age is associated with a higher percentage of aneuploidy in mammalian oocytes (7, 8). The consequence at the end is a decreased fertilisation rate, a decrease in preimplantational embryo development and a decrease in embryo-implantation which cumulatively result in a dramatic decrease in pregnancy potential.

There is an increasing need for therapeutic intervention of the increased infertility in women over the age of 37. Obviously sex steroid hormones are not suitable to positively influence oocyte aging and gonadotropins are more likely to be associated with precocious aging of the oocyte (7). Therefore, compounds are needed that increase the pregnancy potential of older women.

The present invention provides the use of meiosis-activating substances in a culture medium for in vitro fertilization of aged oocytes. Meiosis-activating substances are FF-MAS (follicular fluid meiosis activating sterol) or MAS analogues. FF-MAS is 4,4-dimethyl-5α-cholesta-8,14,24-triene-31-ol. Its synthesis is described in WO99/52930. MAS analogues and their synthesis are described in e.g. WO 96/00235, WO 96/27658, WO97/00884, WO98/28323, WO98/52965 and WO 98/55498. MAS analogues have a percentage germinal vesicle breakdown (GVB) which is significantly higher than the control without FF-MAS when tested in an assay described in example 1. Preferred MAS analogues are such having a percentage GVB of at least 30%, preferably at least 50%. The sterol FF-MAS from human follicular fluid has recently been identified as a compound that induces nuclear (meiotic) and cytoplasmic maturation in the mouse (6, 7, 9).

It has now surprisingly been found that meiose-activating compounds have a significant effect on aged oocytes. Aged oocytes are oocytes of women who are older than 35 years preferably older than 37 years. When meiose-activating compounds are added to a culture medium which is then used for in-vitro-fertilization (IVF) a higher fertililization rate is observed.

The IVF procedure is performed in a known manner. More details about the removal of the oocytes from follicles in the ovary, culturing of the isolated oocytes, the culture medium to be used, the fertilisation with sperm and the transfer of the embryo to the fallopian tube can be found in the literature, e.g. in U.S. Pat. No. 5,693,534 which is hereby incorporated by reference.

A further object of the present invention is the use of compounds that lead to an increase in the FF-MAS concentration in the oocyte in a culture medium for in vitro fertilization of aged oocytes. The synthesis of FF-MAS from lanosterol is catalysed by cytochrome P450 lanosterol 14alpha-demethylase. FF-MAS is converted to T-MAS by the activity of sterolΔ14-reductase (Δ14R). There are drugs such as AY9944 which act as competitive inhibitors of Δ14R (11). Recently it could be shown in the mouse cumulus-oocyte complex (COC) that addition of AY9944 to the culture medium has the capacity to induce accumulation of FF-MAS (12). By increasing the endogenous concentration of FF-MAS, these compounds have the same effect as FF-MAS itself.

The endogenous production of MAS in the intact cumulus enclosed oocyte is also enhanced by growth factors such as epidermal growth factor (EGF) (14). Growth factors have been shown to enhance oocyte maturation in a number of species like the cow, pig, mouse and rat and human (13). Recently, it has been shown that addition of EGF—in concentrations around those observed in vivo—to the culture medium of human oocytes during the cause of IVF treatment results in significant improved implantation rates of the retrieved oocyted (14). These growth factors can be used in a culture medium for in vitro fertilization of aged oocytes.

In a further embodiment the invention provides the use of meiosis-activating substances for the preparation of a medicament for use in the fertilization of aged oocytes. A woman in need of such treatment may be treated with the medicament by intravenous, subcutaneous, oral, intranasal, transdermal, intrauterine, intracervical or intravaginal administration. Usually, not more than 1000 mg, preferably not more than 100 mg, and in some preferred instances not more than 10 mg of FF-MAS and/or one or more MAS analogues is to be administered to women per day. Treatment may either be continuously or intermittent. The medicament may further comprise pharmaceutically acceptable excipients well known in the art like carriers, diluents, absorption enhancers, preservatives, buffers, agents for adjusting the osmotic pressure, tablet disintegrating agents and other ingredients which are conventionally used in the art.

The invention provides further a method of increasing maturation and fertilization rate of aged oocytes which method comprises administering a meiosis-activating substance to an aged oocyte.

### EXAMPLES

The present invention will be illustrated in detail in the following examples.

### EXAMPLE 1

### Method Used for Electing MAS Compounds

Oocytes were obtained from immature female mice (C57BI/6J×DBA/2J F1-hybrids, Bomholtgaard, Denmark) weighing 13-16 grams, that were kept under controlled lighting and temperature. The mice received an intra-peritoneal injection of 0.2 ml gonadotropins (Gonal F, Serono, Solna, Sweden, containing 20 IU FSH, alternatively, Puregon, Organon, Swords, Ireland containing 20 IU FSH) and 48 hours later the animals were killed by cervical dislocation.

The ovaries were dissected out and the oocytes were isolated in Hx-medium (see below) under a stereo microscope by manual rupture of the follicles using a pair of 27 gauge needles. Spherical, naked oocytes (NO) displaying an intact germinal vesicle (GV) were placed in α-minimum essential medium (α-MEM without ribonucleosides, Gibco BRL, Cat. No. 22561) supplemented with 3 mM hypoxanthine (Sigma Cat. No. H-9377), 8 mg/ml Human Serum Albumin (HSA, State Serum Institute, Denmark), 0.23 mM pyrubate (Sigma, Cat. No. S-8636), 2 mM glutamine (Flow Cat. No. 16-801), 100 IU/ml penicillin and 100 μg/ml streptomycin (Flow, Cat No. 16-700). This medium was designated Hx-medium.

The oocytes were rinsed three times in Hx-medium and cultured in 4-well multidishes (Nuncion, Denmark) in which each well contained 0.4 ml of Hx-medium and 35-45 oocytes. One control (i.e. 35-45 oocytes cultured in Hx-medium with no addition of test compound) was always run simultaneously with the test cultures, which were made with different concentrations of the compounds to be tested.

The cultures were performed at 37° C. and 100% humidity with 5% CO₂ in air. The culture time was 22-24 hours.

By the end of the culture period, the number of oocytes with germinal vesicle (GV) or germinal vesicle breakdown (GVB) and those with polar body (PB) was counted using a stereo microscope or an inverted microscope with differential interference contrast equipment. The percentage of oocytes with GVB per total number of oocytes and the percentage of oocytes with PB per total number of oocytes was calculated in the test cultures and compared to the control culture.

% GVB=((number of GVB+number of PB)/total number of oocytes)×100

### EXAMPLE 2

### Effect of Meiosis-Activating Substances on Aged Oocytes

The CBA/Ca mouse provides a model system for studies on the maternal age effect of oocytes (7). This model is characterised by its relatively limited pool of follicles with low number of aged oocytes in the sexually mature adult female, similar to the human. All the following studies were carried out in the CBA/Ca mice. Animals were kept under controlled lighting and temperature.

Collection and Culture of Oocytes

Oocytes from young (5-6 weeks old) and aged (35-38 week old) CBA/Ca mice in the diestrus stage of the cycle were investigated. Oocytes were isolated in Hx-medium (see below) under a stereo microscope by manual rupture of the follicles using a pair of 27 gauge needles. Spherical, naked oocytes (NO) displaying an intact germinal vesicle (GV) were placed in a-minimum essential medium (x-MEM without ribonucleosides, Gibco BRL, Cat. No. 22561) supplemented with 3 mM hypoxanthine (Sigma Cat. No. H-9377), 8 mg/ml Human Serum Albumin (HSA, State Serum Institute, Denmark), 0.23 mM pyruvate (Sigma, Cat. No. S-8636), 2 mM glutamine (Flow Cat. No. 16-801), 100 IU/ml penicillin and 100 μg/ml streptomycin (Flow, Cat No. 16-700). This medium was designated Hx-medium.

The oocytes were rinsed three times in Hx-medium and cultured in 4-well multidishes (Nuncion, Denmark) in which each well contained either (a) hypoxanthine (Hx)-free medium, (b) 10 μM FF-MAS in Hx-free medium, (c) Hx-medium or (d) 10 μM FF-MAS in Hx-medium. The cultures were performed at 37° C. and 100% humidity with 5% CO₂ in air. The culture time was 22 hours.

Evaluation of Oocytes

(a) By the end of the culture period cell cycle progression was analysed by counting the number of oocytes with germinal vesicle (GV) or germinal vesicle breakdown (GVB) and those with polar body (PB) by using a stereo microscope or an inverted microscope with differential interference contrast equipment. The percentage of oocytes with GV, GVBD and PB per total number of oocytes, respectively, was calculated in the 4 treatment groups of young and aged oocytes, respectively.

(b) Cytogenetic analysis was performed at the end of the experiment with all oocytes by using the modified spreading and banding technique by Eichenlaub-Ritter & Betzendahl (15). This technique yielded very clear chromosome preparations. The number of chromosomes was counted in each oocyte. Oocytes with a number of 20 chromosomes were defined as euploid oocytes. Oocytes with a number of more or less than 20 chromosomes were defined as aneuploid oocytes. Aneuploid oocytes with more than 20 chromosomes were defined as hyperploid, those with a number of less than 20 chromosomes as hypoploid.

Results

(a) FF-MAS+Hx treated aged oocytes are more successful to mature to metaphase 11 than young oocytes (FIG. 1)

(b) FF-MAS reduces numerical aberration rate, e.g. hyperploidy in aged metaphase 11 oocytes (FIG. 2) when given alone or in combination with Hx

### DESCRIPTION OF THE FIGURES

FIG. 1 shows the cell cycle progression of young and aged oocytes

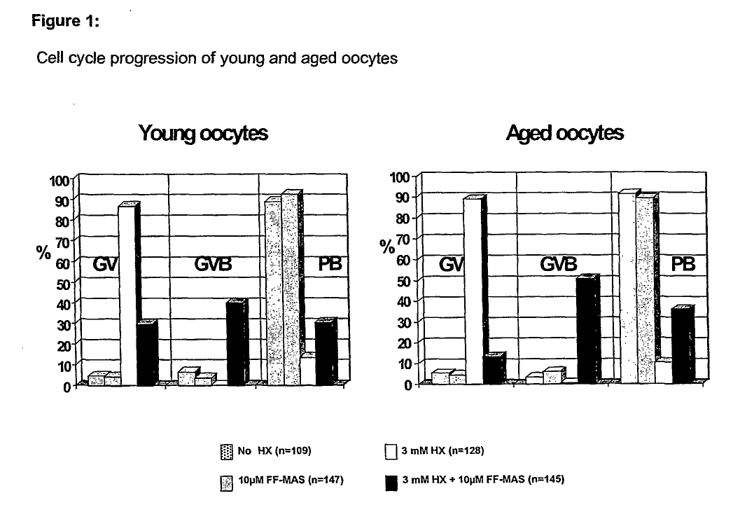

FIG. 2 shows the hyperploidy rate [%] of young and aged oocytes.

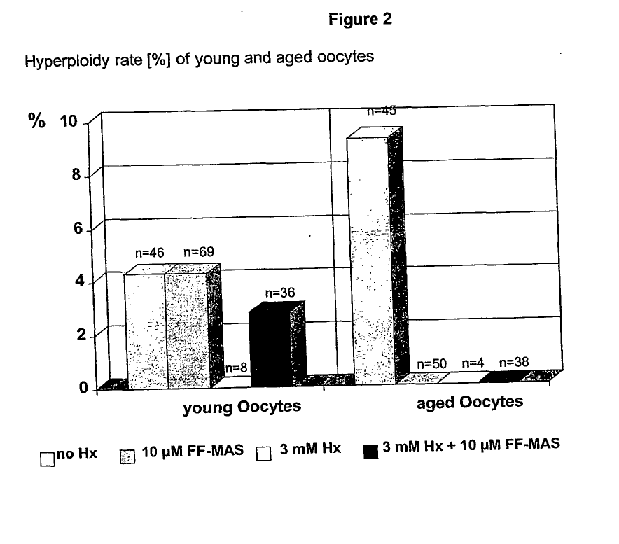

### REFERENCES

1. National Center for Health Statistics (1993), Mon Vital Stat Rep 32(9)

2. Office of Population Censuses and Surveys (1991). Birth Statistics 1990 Series Fm No. 19. HM Stationery Office. London.

3. Ventura S J et al. (1994) Mon Vital Stat Rep 43: 1-88

4. Sauer M V (1994) In: Treatment of the postmenopausal woman: Basic and clinical aspects. Lobo RA (ed.), Raven Press, New York, pp. 35-46

5. Faddy M J R G et al. (1992) Hum Reprod 7: 1342-1346

6. Gosden R G (1997) Hum Reprod 12:1

7. Eichenlaub-Ritter (1998) Maturitas 30: 143-169

8. Plachot M A et al. (1988) Hum Reprod 3: 627-635

9. Hegele-Hartung et al. (1999) Biol Reprod

10. Hegele-Hartung et al. (1998) Hum Reprod 13:98

11. Mercer (1993) Prog Lipid Res 32: 357-416

12. Leonardsen et al. (2000) J Reprod Fertil 118: 171-179

13. Goud et al. (1998) Hum Reprod 13: 1638-1644

14. Byskov et al. (1999) J Exp Zool 285: 237-242

15. Eichenlaub-Ritter and Betzendahl (1995) Mutagenesis 10: 477-486

1. Use of meiosis-activating substances in a culture medium for in vitro fertilization of aged oocytes.

2. Use of claim 1 wherein the meiosis-activating substance is FF-MAS.

3. Use of claim 1 wherein the meiosis-activating substance is a MAS analogue.

4. Use of compounds that lead to an increase in the FF-MAS concentration in the oocyte in a culture medium for in vitro fertilization of aged oocytes.

5. Use of claim 4 wherein the compounds that lead to an increase in the FF-MAS concentration in the oocyte is a A14 reductase inhibitor.

6. Use of claim 4 wherein the compounds that lead to an increase in the FF-MAS concentration in the oocyte is a growth hormone.

7. Use of claim 4 wherein the compounds that lead to an increase in the FF-MAS concentration in the oocyte is EGF.

8. Use of meiosis-activating substances for the preparation of a medicament for use in the fertilization of aged oocytes.

9. Use of compounds that lead to an increase in the FF-MAS concentration in the oocyte for the preparation of a medicament for use in the fertilization of aged oocytes.

10. A method of increasing maturation and fertilization rate of aged oocytes which method comprises administering a meiosis-activating substance to an aged oocyte.
